(19) United States
(12) Patent Application Publication
(10) Pub. No.: US 2026/0198947 A1
(43) Pub. Date: Jul. 16, 2026

(54) INTRAVASCULAR LITHOTRIPSY DEVICES AND SYSTEMS WITH FORWARD FACING ELECTRODES AND FLEX CIRCUIT ARRANGEMENTS

(71) Applicant: Cardiovascular Systems, Inc., St. Paul, MN (US)

(72) Inventors: Matthew W. Tilstra, Rogers, MN (US); Joseph P. Higgins, Wayzata, MN (US)

(21) Appl. No.: 19/120,110

(22) PCT Filed: Oct. 12, 2023
(86) PCT No.: PCT/US2023/035025

§ 371 Date: Apr. 10, 2025

Related U.S. Application Data
(60) Provisional application No. 63/416,231, filed on Oct. 14, 2022.
(60) Provisional application No. 63/462,208, filed on Apr. 26, 2023.

(51) Int. Cl. A61B 17/22; A61B 17/00

(52) U.S. Cl. CPC A61B 17/22022; A61B 2017/00778; A61B 2017/22008; A61B 2017/22062

(57) ABSTRACT

A catheter system creates a primarily forward force from forwardly facing electrodes provided within the arrangement of a balloon catheter. The system includes a high voltage pulse generator that provides plus and minus voltage connections with electrode wires. The electrode wires can also pass through a lumen of a catheter toward a distal end of the catheter where they are connected to electrodes preferably arranged in series to create one of more energy waves for propagation toward a thrombus or calcified lesion. Inflation fluid can be injected to balloon as facilitated. The inflation fluid is preferably a saline solution so that it has some level of conductivity.

### CROSS-REFERENCE TO RELATED APPLICATIONS

This application claims priority to and the benefit of U.S. Provisional Application No. 63/416,231, filed Oct. 14, 2022, and entitled CATHETER SYSTEM WITH FORWARD FACING ELECTRODES FOR CREATING ENERGY WAVES, and U.S. Provisional Application No. 63/462,208, filed Apr. 26, 2023 and entitled INTRAVASCULAR LITHOTRIPSY DEVICES AND SYSTEMS WITH FORWARD FACING ELECTRODES AND FLEX CIRCUIT ARRANGEMENTS, the entire contents of which are incorporated herein by reference in their entireties.

### TECHNICAL FIELD

The present invention is directed to a catheter system for treating a vascular thrombus or calcified lesion or the like utilizing energy waves generated by electrodes within a conductive fluid medium.

### BACKGROUND

Catheter systems having an angioplasty balloon are commonly used to apply a physical force by expansion of the balloon against a calcified lesion within vasculature to force the calcification back into and against the blood vessel wall. Certain such calcified lesions and thrombi are not effectively broken up by the use of an angioplasty balloon. For example, a thrombus may be present within the vasculature in front of an inserted angioplasty balloon. Angioplasty balloons do not work effectively against a forward obstruction.

More recently, intravascular lithotripsy (IVL) catheter systems have been developed that include a balloon similar to an angioplasty balloon that is filled with a conductive liquid medium, such as a saline solution, for expanding the balloon in position at the lesion or thrombus, wherein the catheter system includes a pair of electrodes operatively positioned within the conductive liquid medium. The electrodes are pulsed with high voltage so as to create a spark that jumps over a gap between the two electrodes at each pulse. The spark within the conductive medium creates an energy wave that propagates through the liquid medium causing the balloon to physically provide a force against the lesion or thrombus. The energy propagation includes the creation of micro-bubbles that also facilitate the physical force. Such devices are known to provide a primarily radial energy wave to act against a lesion or thrombus radially for purposes of breaking up the calcification or clotting.

Current catheter systems include a therapy sequence that includes a maximum number of continuous pulses, followed by a minimum delay time and a hard maximum total pulses associated with a particular catheter. One such product specifies:

| Treatment Frequency | 1 Hz (1 Pulse | |
|---|---|---|
| | per Second) | |
| Maximum Number of Continuous | 30 | Pulses |
| Pulses (1 cycle) | | |
| Minimum Pause Time | 10 | Seconds |
| Maximum Total Pulses | 300 | Pulses |
| Per Catheter | | |

If a therapy is not yet completed after the maximum total pulse per catheter count, the physician must replace the catheter with the attendant undesirable, cost, delay and distraction.

Intravascular lithotripsy (IVL) devices are available for some calcification patterns. Disposable IVL balloon devices are provided in different designs and sizes for peripheral or coronary indications. All designs utilize a reusable power source such as an IVL generator. One reusable DC generator comprises the following specification:

| | |
|---|---|
| Power | 110-240 VAC; 50-60 Hz; Single Phase, 15 A |
| | service |
| Size | 11″ (28.0 cm) high × 6″ (15.2 cm) wide × |
| | 11.5″ (29.2 cm) deep |
| Weight | 15 pounds (6.8 kg) |
| Output | Proprietary pulse delivery system. Output |
| | voltage 3000 volts peak, pulse |
| | frequency 1 Hz |
| Mobility | Product is designed to be mounted to an IV |
| | pole |
| Length | 5 ft (1.53 m) |
| Compatibility | Male key distally designed to connect only |
| | to catheter. |
| Operation | Lithotripsy pulsing is activated by pushing a |
| | button on the Connector Cable. |
| Use | Re-usable |

One such disposable device consists of a 0.014-inch guidewire-compatible, fluid-filled balloon angioplasty catheter with two lithotripsy emitters incorporated into the shaft of the 12-mm-long balloon segment. A fluid filled balloon (e.g. a 50/50 saline contrast medium) is inflated to about 4 atm and then electrical pulses are provided to the emitters that create high voltage sparks to provide the therapy. Acoustic waves are created and the calcium is fractured.

### SUMMARY

The present invention is directed to a catheter system that creates a primarily forward force from forwardly facing electrodes provided within the arrangement of a balloon catheter. The system includes a high voltage pulse generator that provides plus and minus voltage connections with electrode wires. The electrode wires can also pass through a lumen of a catheter toward a distal end of the catheter where they are connected to electrodes preferably arranged in series to create one of more energy waves for propagation toward a thrombus or calcified lesion. Inflation fluid can be injected to balloon as facilitated. The inflation fluid is preferably a saline solution so that it has some level of conductivity.

In one aspect of the present invention, an intravascular lithotripsy (IVL) system is provided for use in creating an energy wave as a force to a lesion within a vasculature, the IVL system comprising, a catheter that extends from a proximal end to a distal end with an electrode and conductive tube arrangement at a distal end of the catheter, wherein the electrode is provided within the conductive tubing and spaced from the conductive tubing by an insulating layer, the electrode having a forward-facing electrode distal end, the conductive tubing also including a forward-facing conductive tubing distal end that is spaced radially relative to the distal end of the electrode to create at least one spark gap between the forward facing electrode distal end.

The IVL system may include plural electrodes within the conductive tubing, each spaced from the conductive tubing by the insulating layer and with the electrodes being electrically isolated from one another so as to create plural gaps with the conductive tubing that can be energized in series to create plural sparks. Moreover, The electrodes can be arc segments of conductive material that as spaced similarly from the conductive tubing are coaxial with the conductive tubing, and the distal ends of the electrodes and the distal end of the conductive tubing extend to a similar axial length so as to terminated adjacent to one another.

In another aspect, the IVL system may have the distal end of the conductive tubing extended axially more distally than the distal end of the electrode so that the entire distal end of the electrode is proximally positioned within the conductive tubing and spaced from the distal end of the conductive tubing. The insulating layer can preferably extend to terminate adjacent to the distal end of the electrode so that a spark can be generated between the distal end of the electrode and an inside side wall of the conductive tubing.

In another aspect of the present invention, a method of using an intravascular lithotripsy (IVL) system against a lesion within a vasculature can comprise inserting an IVL catheter having a balloon, a conductive tubing, and at least one electrode within the vasculature of a patient to the point of a lesion within the vasculature being located beyond the distal end of the balloon, wherein the electrode has a distal end and the conductive tube also has a distal end so that a spark gap is created between the distal ends of the electrode and the conductive tubing; electrically connecting the conductive tubing and the electrode with a high voltage pulse generator; delivering a fluid that is at least partially conductive to the balloon; and generating a high voltage pulse at the high voltage pulse generator and thereby creating a spark at the spark gap with a cavitation bubble such that forward-facing energy waves are propagated within the fluid of the balloon and from the balloon to the lesion.

Such an IVL system can have plural electrodes within the conductive tubing, each spaced from the conductive tubing by the insulating layer and with the electrodes being electrically isolated from one another so as to create plural spark gaps with the conductive tubing, the method further comprising energizing the electrodes in series to create plural sparks at the plural spark gaps. The distal ends of the electrodes and the distal end of the conductive tubing can extend axially a similar distance and terminate adjacent to one another with the insulating layer between them so that the spark can be generated from the distal end of the electrodes and the distal end of the conductive tubing. Otherwise, the distal end of the conductive tubing can extend axially more distally than the distal end of the electrode so that the entire distal end of the electrode is proximally positioned within the conductive tubing and spaced from the distal end of the conductive tubing, the method further comprising generating a spark between a distal end of the electrode and an inside side wall of the conductive tubing and thus generating a cavitation bubble at least partially within the distal end of the conductive tubing so that energy waves can be directed from an open distal end of the conductive tubing in a desired forward direction. More preferably, the distal end of the conductive tubing can extend sufficiently axially beyond the distal end of the electrode so that the entire cavitation bubble is formed within the distal end of the conductive tubing.

In another aspect, an intravascular lithotripsy (IVL) system for use in providing an energy wave as a force to a lesion with a vasculature, the IVL system includes a catheter that extends from a proximal end to a distal end with an electrode and conductive tube arrangement at a distal end of the catheter with plural electrodes provided adjacent to the distal end of the catheter, also including a flex circuit extending from the proximal end of the catheter to the electrodes for electrically connecting the electrodes to a high voltage pulse generator at the proximal end of the flex circuit, wherein the flex circuit is spirally wound within and along at least a portion of the catheter.

### BRIEF DESCRIPTION OF THE DRAWINGS

FIG. 1 is a schematic drawing of a high voltage pulse generator along with a balloon catheter and including an arrangement of electrodes and a conductive tube for creating forward facing (in the axial direction) sparks as an Intravascular lithotripsy (IVL) device;

FIG. 2 is a schematic view similar to FIG. 1, but with an open-ended balloon to permit conductive fluid flow from the balloon during an IVL procedure;

FIG. 3 is a perspective view of a distal portion of a conductive tube within which a pair of arcuate electrodes are positioned to create and IVL device without the insulation layers shown;

FIG. 4 is a cross-sectional view of a distal portion of an IVL device showing the ends of two electrodes and conductive tube and where the sparks will generate and cause an axially-forward direction of energy propagation;

FIG. 5 is a cross-sectional view similar to FIG. 4 having the conductive tube extending further in the axially forward direction than the ends of the electrodes so as the create a slightly radially inward propagation of energy from generated sparks;

FIG. 6 shows a distal portion of the IVL device of FIGS. 1-5 in cross-section illustrating the conductive electrodes and conductive tube insulated from one another;

FIG. 7 is a cross-section of a flex circuit portion comprising an insulating layer with plural electrodes to be rolled and inserted within a conductive tube to provided an electrode structure in accordance with the present invention;

FIG. 8 is a cross-section of a resultant electrode arrangement comprising the flex circuit of FIG. 7;

FIG. 9 is another flex circuit arrangement comprising a conductive layer, an insulating later and plural electrodes suitable for creating an electrode structure of the present invention;

FIG. 10 is a cross-section of a resultant electrode arrangement comprising the flex circuit of FIG. 9 as wrapped about a lumen defining tube;

FIG. 11 is a similar flex circuit as shown in FIG. 9 but that is shortened so as not to be as long as a circumference of a lumen defining tube;

FIG. 12 is a cross-section of the flex circuit wrapped partially around the lumen defining tube;

FIG. 13 is a view of a flex circuit portion having plural electrode pads connected with traces for electrical connection and energization;

FIG. 14 is similar to FIG. 13 and showing sparks generated from the electrode pads to a conductive outer layer;

FIG. 15 is a distal portion of a electrode pad connected with a trace that is angled to provide for a spiral winding of the trace to extend from the electrode pad for running the length of the catheter;

FIG. 16 is a length of such an electrode pad and angled trace as suitable to be wound as shown in FIG. 15;

FIG. 17 is a distal portion of an electrode with plural traces running from the electrode pad and that can also be wound as extended;

FIG. 18 is a view similar to FIG. 16 illustrating a pair of electrical conductors provided along one side of a single trace for connecting with a pair of electrode pads;

FIG. 19 is an enlarged view of the distal end portion of the trace, conductors, and electrode pads of FIG. 18;

FIG. 20 is a view similar to FIG. 18 illustrating a pair of electrical conductors provided along both sides of a single trace for connecting with a pair of electrode pads;

FIG. 21 is an enlarged view of the distal end portion of the trace, conductors, and electrode pads of FIG. 20;

FIG. 22 is a view of a length of a pair of electrodes with a trace having a pair of conductors extended linearly for connection with a high voltage pulse generator;

FIG. 23 is an enlarged distal portion of the electrodes, conductors, and trace of the flex circuit of FIG. 22;

FIG. 24 is an axial cross-sectional view similar to FIG. 5 having the conductive tube extending further in the axially forward direction than the ends of the electrodes so as the create a spark between an end of an electrode and an inside wall of the conductive tube for energy wave propagation from the end of the conductive tube; and

FIG. 25 is traverse cross-sectional view of the conductive tube and electrode arrangement of FIG. 24 illustrating a single electrode as a coaxial conductive tube within an outer conductive tube.

### DETAILED DESCRIPTION

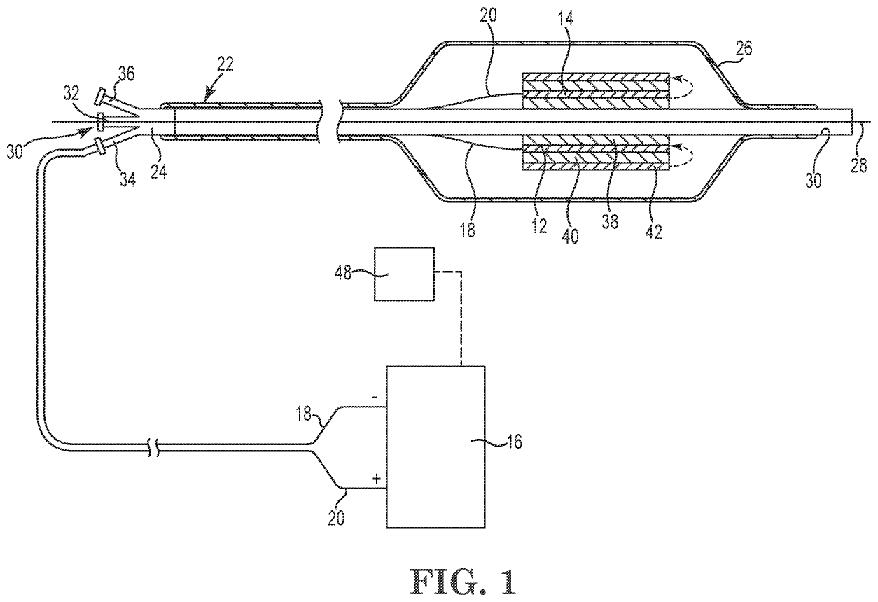

The present invention is directed to a catheter system 10 as schematically illustrated in FIG. 1 that creates a primarily forward force from forwardly facing electrodes 12 and 14 described as follows. The system 10 includes a high voltage pulse generator 16 that provides plus and minus voltage connections with electrode wires 18 and 20. A catheter 22 includes a hub 24 at a proximal end and a balloon 26 at a distal operative end. A guide wire 28 slides within a lumen 30 that extends from an access portal 32 of the hub 24 to the distal end of the balloon 26. The electrode wires 18 and 20 can also pass through the lumen 30 toward the distal end of the catheter 22 by way of an electrical portal 34 of the hub 24. Inflation fluid can be injected to the balloon as facilitated by an inflation portal 36, with the fluid flow through the catheter 22 being provided by any inflation lumen as may be provided to the balloon 26 as well known. The inflation fluid is preferably a saline solution so that it has some level of conductivity for purposes as described below.

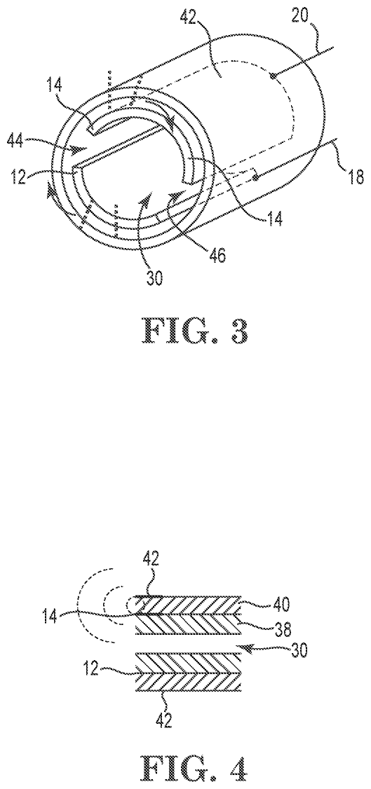

The electrode wires 18 and 20, as shown within the balloon 26, are electrically connected with electrodes 12 and 14, respectively. The electrode 12 is preferably spaced from the wire lumen 30 by a first insulating layer 38 that surrounds and can define the lumen 30. A second insulating layer 40 preferably surrounds the electrodes 12 and 14 so as to electrically insulate the electrodes 12 and 14 from a conductive tube 42. As best shown schematically in FIG. 3, each electrode 12 and 14 can be preferably provided within the same radial space from the lumen 30. As shown, the electrode 12 is arcuate in shape as viewed from either its distal or proximal end and comprises an arc segment, preferably less than 180 degrees and extends axially over a predetermined length and is connected with electrode wire 20. Likewise, the electrode 14 is arcuate in shape as viewed from either its distal or proximal end and comprises another arc segment, preferably also less than 180 degrees, and that extends axially over a predetermined length and is connected with electrode wire 18. The electrodes 12 and 14 can be of similar or different lengths. The length of the electrodes 12 and 14 can relate to the useful life of them, as they will wear down over time as described in more detail below. The conductive tube 42 is schematically positioned as surrounding both electrodes 12 and 14 in a preferably concentric manner.

Not shown in FIG. 3 are the insulating layers 38 and 40. Layer 38 would be between the lumen 30 and the electrodes 12 and 14, preferably also as a concentric layer. Layer 40 would be between the electrodes 12 and 14 and the conductive tube 42, preferably also as a concentric layer. Whereas the electrodes 12 and 14 are each less of an arc segment than 180 degrees, gaps 44 and 46 are formed between respective axial edges of each side of both electrodes 12 and 14. Preferably, the gaps are similar in arc segment lengths, but need not be. In order to prevent any arcing between the electrodes 12 and 14 at the gaps 44 and 46, insulation material from either insulating layer 38 or 40 or both is preferably also provided to fill the gaps 44 and 46.

Referring back to FIG. 1, the distalmost end of the electrodes 12 and 14 is where electrode sparking is to be controllably performed by high voltage pulses created by the generator 16. A controller 48 is shown schematically in operative connection with the high voltage pulse generator 16 and can include an operator input module so that an operator can control to pulses and thus sparking across the electrodes 12 and 14 and the conductive tube 42. The controller 48 also can include programming to control the high voltage pulses according to a predetermined sequence that can be tailored for a specific vasculature situation or other circumstances.

For each high voltage pulse, plural sparks are preferably created. In the example of FIGS. 1-5, a positive charge can be provided to the electrode 14 by electrode wire 20 from the high voltage pulse generator 16 as a single pulse or a series of controlled pulses. A neutral or ground can be applied from the generator 16 to electrode 12 by way of electrode wire 18. As a result, for each pulse a spark will arc from the distal end surface of electrode 14 to the distal end surface of the conductive tube 42. That charge provided to the conductive tube 42 will create a second spark as an arc from the conductive tube 42 to the neutral electrode 12. In this manner, the spark gaps are electrically connected in series. As above, the balloon 26 is filled with a fluid medium during this process, wherein the fluid medium is at least partially conductive, such as a saline solution. Other fluid mediums as known or developed can also be used. The dashed arrows in FIG. 1 at the distal ends of the electrodes 12 and 14 and the conductive tube 42 show the electrical arcing as described. In FIGS. 3 and 4, the propagation of energy is illustrated by dashed lines.

In accordance with a preferred aspect of the present invention, the sparks or electrical arcing at each occurrence is from a forward-facing surface of each of the electrodes 12 and 14 and the conductive tube 42. More preferably, the forward-facing surfaces are at the distal end of the electrodes 12 and 14 and the conductive tube 42. In order to prevent sparking at the proximal side of the electrodes, an insulator material can also be applied covering both proximal or rearward-facing surfaces of the electrodes 12 and 14. As a result of such orientation, each spark produces an energy wave that propagates in a forward direction, as defined as a primarily axial direction from the distal end of the electrodes 12 and 14 and the conductive tube 42. In operation, the catheter 22 would be inserted within the vasculature so that the balloon 26 abuts against a thrombus or other calcified lesion with a distal portion of the balloon 26 likely deforming against such thrombus or lesion. High voltage pulsing would thus create one or a series of energy waves that propagate through the fluid medium within the balloon 26 in the forward direction to apply a force or a series of forces against the thrombus or lesion for breaking it up.

Upon each firing and sparking between the electrodes 12 and 14 and the conductive tube 42, a bit of the respective forward-facing surfaces of each will disintegrate at the closest points. As such, the sparks will move from one point to a new closest point or smallest gap between these forward-facing surfaces. The sparks will thus travel along the arcuate forward-facing surfaces of the electrodes 12 and 14 and the conductive tube 42 between the axial edges of the electrodes 12 and 14. Over time, the electrodes 12 and 14 and the conductive tube 42 will wear both along the arcuate forward-facing surfaces of the electrodes 12 and 14 and the conductive tube 42 as well as axially. The useful life of the electrodes 12 and 14 and the conductive tube 42 can be based on the axial length of wear that is determined to be acceptable.

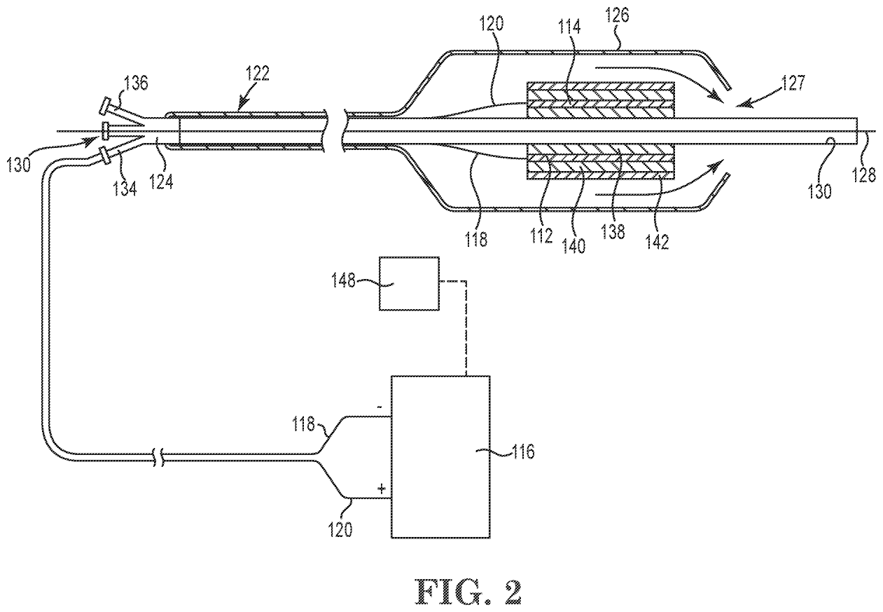

FIG. 2 illustrates a catheter embodiment that is substantially similar to that of FIG. 1, but that a distal end of the balloon 126 is open as indicated at 127 through which fluid medium can pass. In operation, fluid would be supplied under some pressure to cause an axial flow from the distal end of the balloon 126 (fluid flow shown by arrows) while the sparks and energy waves are generated from the high voltage pulses. Such fluid flow can be helpful in breaking up a thrombus or lesion. The fluid flow can be controlled to be minimal, such as having to weep through the open end at 127, or the fluid flow can be controlled to supplement the impact to the thrombus or lesion. In this case, microbubbles within the fluid as created by an energy wave can pass through the open end 127 to interact with the thrombus or lesion. All other components and features, as discussed with reference to FIGS. 1, 3 and 4 are similar but labelled with similar numbers with a 1 in the hundred position.

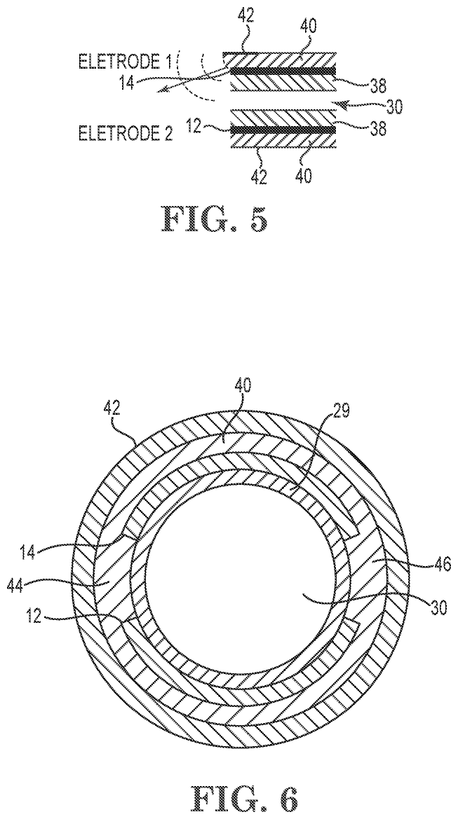

Another example of a forward-facing electrode arrangement is shown in FIG. 5 for generating an energy wave that propagates in a primarily axial or forward direction. In this example, the distal end and thus the forward-facing surface of at least a portion of the conductive tube 42 is extended axially beyond the forward-facing surface of electrode 14. The axial distance that the conductive tube extends beyond the forward faces of the electrodes 12 and/or 14 can vary depending on the directional control desired of the sparks. The insulation layer 40 can also extended and sloped from the distal tip of the conductive tube 42 to the electrode 14, although it need not be. The sparks could generate between the ends of the conductive tube 42 and electrodes 12 and 14, as above, or the sparks could generative from an inside surface of the conductive tube slightly proximal from its distal end. Sparks will be generated with this arrangement in a primarily forward and axial direction, but the energy waves will propagate also somewhat radially inward, as illustrated by the wave pattern. It is contemplated that the entire circumference of the conductive tube 42 can be extended distally forward as compared to both electrodes 12 and 14 so that both sparks will propagate a similar wave pattern that is primarily forward and somewhat radially inward. It is also contemplated that an opposite arrangement can be done. One or both of the electrodes 12 and/or 14 can be extended forward of the distal end of the conductive tube 42. In such an arrangement, one or both energy waves can be propagated in a primarily forward or axial direction while also being somewhat radially outward. One energy wave can be primarily forward and radially inward combined with another energy wave that is primarily forward and radially outward. Also, any combination of a forward energy wave, as above, can be combined with another energy wave of primarily forward and radially inward or outward.

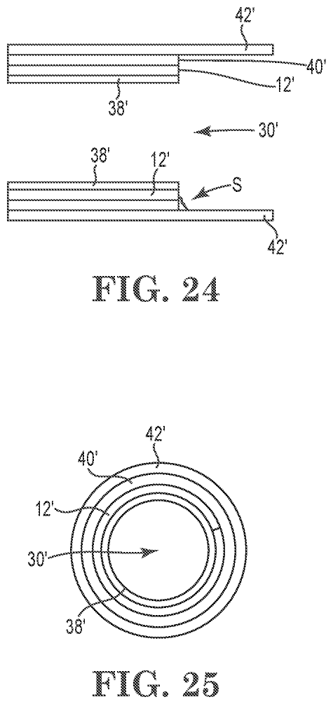

Yet another example of a forward-facing electrode arrangement is shown in FIGS. 24 and 25 also for generating an energy wave that propagates in a primarily axial forward direction. In this arrangement, an outer conductive tube 42′ extends axially further distally than a single inner tubular electrode 12′. An insulating layer 40′ preferably extends to a similar extent as the electrode 12′. Likewise, an insulating layer 38′ preferably also extends to a similar extent as the electrode 12′ and creates a lumen 30′ for a guidewire. The single electrode 12′ is shown as a conductive tube that is concentric with the outer conductive tube 42′ and would be electrically connected with either a positive or negative conductor from the high voltage pulse generator 16 with the outer conductive tube 42′ electrically connected with the other of the positive or negative conductor from the high voltage pulse generator 16. That way, a spark S can be generated between a distal portion of the outer conductive tube 42′ and the electrode 12′. As shown, the spark S and a generated cavitation bubble will preferably occur at least partially within (and more preferably entirely within) the distal end portion of the outer conductive tube 42′ with the spark S between an end surface of the electrode 12′ and a inside wall surface of the outer conductive tube 42′. By this arrangement, a cavitation bubble (or microbubbles) will be generated by the spark S at least partially (and more preferably entirely) within the distal end of the conductive tube 42′, which will directionally guide or manage the released energy as an energy wave in a forward direction out from an opening of the outer conductive tube 42′ and to a lesion. Surprisingly, this may be accomplished without destroying the components of the catheter for most applications. The spark S will create an energy wave preferably at least partially within the distal end of the outer conductive tube 42′ to propagate from the distal end of the outer conductive tube 42′. Also, cavitation of microbubbles within the distal portion of the outer conductive tube 42′ will add to the energy wave created and propagated from the distal end of the conductive tube 42. It is contemplated that plural electrodes can also be used in a similar arrangement, such as a modification of that shown in FIG. 5.

It is also contemplated that more than two such electrodes can be provided. Depending on the number of such electrodes additional conductive tubing may be needed to provide a conductive sequence with controlled sparking at defined gaps.

FIG. 6 illustrates the electrode and conductive tube arrangement of FIGS. 1-4 in cross-section. The conductive tube 42 is illustrated as a seamless tube, also known as a hypotube, that is concentric with a guidewire tube 29 that defines the lumen 30 through which the guidewire 28 can pass. In this arrangement, the tube 29 provides the insulative aspect from one side of the electrodes 12 and 14 without the first insulating layer 38, described above. The electrodes 12 and 14 are positioned within a common radial space and as positioned as arc segments against the outer surface of the tube 29. The second insulating layer 40 is shown between the conductive tube 42 and the outer curved surfaces of the electrodes 12 and 14 as well as filling the gaps 44 and 46 between axially extending edges of the electrodes 12 and 14. Such an arrangement can be made in many ways, such as providing the tube 29 and adhering, welding, or otherwise supporting the electrodes 12 and 14 onto the outer surface of the tube 29. Such a sub-assembly can then be concentrically supported in position to the outer conductive tube 42 followed by injecting an insulating material between the conductive tube 42, the outer surfaces of the respective electrodes 12 and 14, and the outer surface portions of the tube 29 within the gaps 44 and 46.

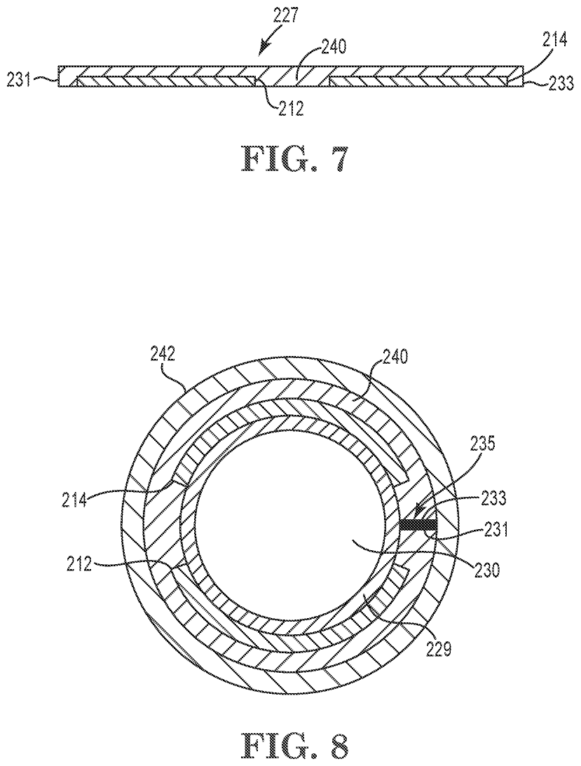

FIGS. 7 and 8 together illustrate a manner of making an electrode and conductive tube arrangement similar to that of FIGS. 1-4. As shown in FIG. 7, electrodes 212 and 214 can be formed along with an insulating layer 240 as a flexible circuit. A flex or flexible circuit 227 can be made by well-known processes that can include material additive or subtractive steps along with masking steps, and controlled deposition and/or etching steps. Flex circuits are well known for combining conductive metal like electrical traces, pads or electrodes with insulating layers and potentially other support materials and that are used for electrical interconnecting of components. In this example, the insulating layer 240 and electrodes 212 and 14 are formed as a flat flex circuit 227 sub-assembly that can then be wrapped about a lumen 230 defining tube 229, adhered, welded or otherwise attached to the tube 229, and then inserted within a conductive tube 242. The conductive tube 242 can be adhered, welded, or otherwise attached to the flex circuit 227 or not. The electrodes 212 and 214 along with the insulating layer 240 must be sufficiently flexible to be able to be rolled up as shown. The electrodes 212 and 214 are positioned along the face of the insulating layer 230, in flat state, so that when rolled up about the tube 229, they will be positioned as desired, preferably at diametrically opposed positions as illustrated in FIG. 8. The flat flex circuit extends from a first axially extending edge 231 (when rolled) to a second axially extending edge 233. When wrapped around the tube 229, a gap 235 is preferably made between the ends 231 and 233 to ensure proper positioning of the electrodes 212 and 214 and so as not to engage with or interfere with one another.

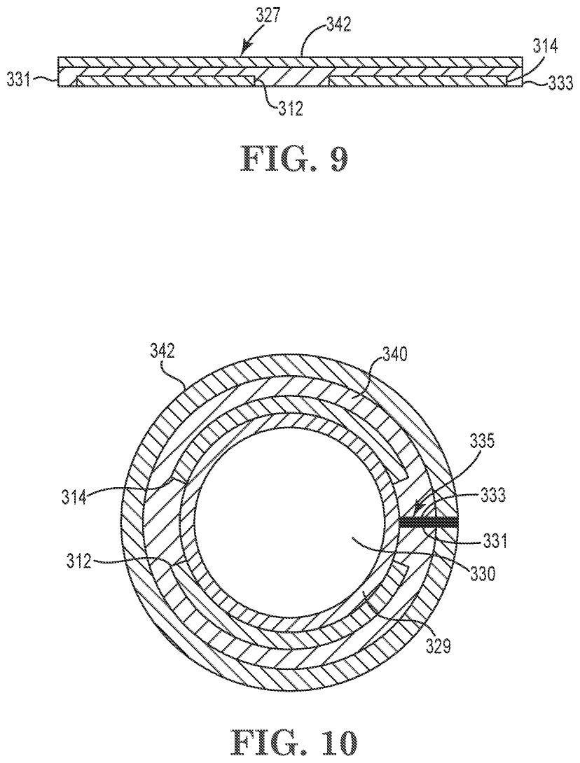

In FIGS. 9 and 10, another manner of making such an electrode and conductive tube arrangement is illustrated. As shown in FIG. 9, an alternative flex circuit 327 is formed comprising a conductive layer 342, an insulating layer 340, and electrodes 312 and 314 embedded within the insulation layer 240. In this case, not only must the electrodes 312 and 314 and insulating layer 340 be sufficiently flexible to roll up, so must also the conductive layer 342. To create the electrode and conductive tube arrangement, the flex circuit 327 of FIG. 9 can be wrapped around the outer surface of tube 329 and secured in place, such as by adhesive, welding, or otherwise. The flat flex circuit 327 extends from a first axially extending edge 331 (when rolled) to a second axially extending edge 333. When wrapped around the tube 329, a gap 335 is preferably made between the ends 331 and 333 to ensure proper positioning of the electrodes 312 and 314 and so as not to engage with or interfere with one another.

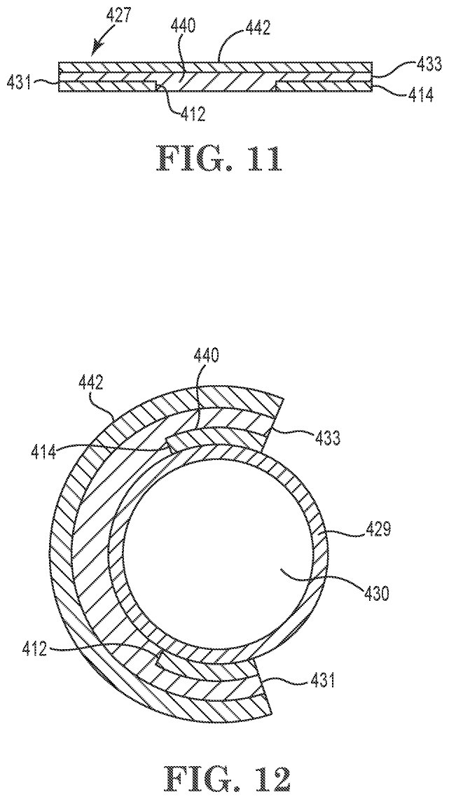

In FIGS. 11 and 12, another manner of making such another electrode and conductive tube arrangement is illustrated. In this arrangement, a flex circuit 427 is made and only partially wrapped around a lumen 430 defining tube 429 and fixed in place. Specifically, a flex circuit is created comprising a conductive layer 442, an insulating layer 440, and electrodes 412 and 414. Also in this arrangement, the electrodes 412 and 414 and insulating layer 440 must be sufficiently flexible to roll up along with the conductive layer 442. To create the electrode and conductive tube arrangement, the flex circuit 427 of FIG. 11 can be partially wrapped around the outer surface of tube 429 and secured in place, such as by adhesive, welding, or otherwise. The flat flex circuit 427 extends from a first axially extending edge 431 (when rolled) to a second axially extending edge 433. When partially wrapped around the tube 429, a gap 435 is preferably made between the ends 431 and 433 to ensure proper positioning of the electrodes 412 and 414 and so as not to engage with or interfere with one another. Such an arrangement can reduce material needs while effectively providing preferably diametrically opposed electrodes 412 and 414 in operative positions relative to the conductive layer 442.

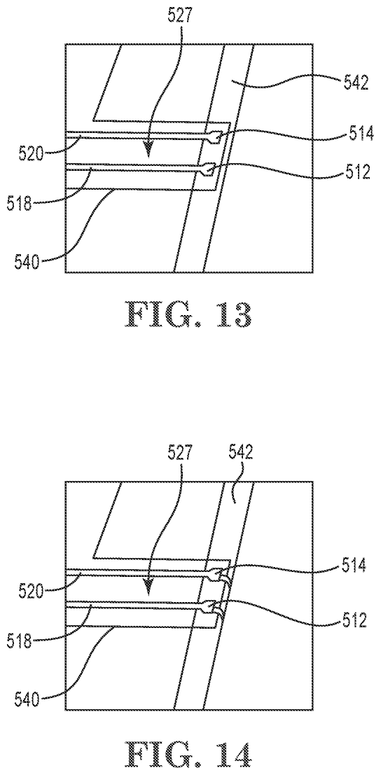

FIGS. 13 and 14 illustrate another advantage of creating an electrode and conductive tube arrangement from a flex circuit. As illustrated, a flex circuit 527 can be made comprising an insulating layer 540 onto which electrical traces 518 and 520 can be created, as described above. The electrical traces 518 and 520 can run axially and terminate at or near the distal end of the insulating layer 540 as pads that are shown as rectangular and that can create electrodes 512 and 514. The flex circuit 527 can be applied by any known bonding technique to a layer of conductive material 542. When rolled up, the electrical traces 518 and 520 run axially of a tube created by rolled up conductive material 542. The flex circuit 527 and conductive material 542 can be wrapped around an outer surface of an insulating tube (not shown, but similar to tube 29) and also bonded in place. The electrical traces 518 and 520 can advantageously extend beyond the proximal end of the insulating layer 540 and/or the conductive material 542, potentially all the way through and outside of the hub 24. With such an arrangement, there would be no need to make any electrical connection within the balloon or anywhere along the catheter. Not making the electrical connection within the balloon would allow for a smaller profile of the balloon and electrode/conductive tube within a patient's body.

FIG. 14 illustrates a sparking between the distal ends of each electrode 512 and 514 and the distal end of the conductive material 542. The spark can be controlled to be more radial or axially, as discussed above, by the relative extension of the electrodes 512 and 514 and the conductive material 542 and the insulating material 540.

Other flex circuits are contemplated that may facilitate easier running of a flex circuit over the length of the catheter to the electrodes and preferably with minimal effect on catheter stiffness so as to allow desired IVL balloon positioning within a patient's vasculature. As above, preferably, two conductors can electrically connect any number of emitters, each comprising two electrodes, in series. Flex circuit designs can thus include two electrical traces on an insulating flexible layer to extend from a high voltage pulse generator to the one of more emitters of an IVL balloon.

The following embodiments of flex circuits in accordance with the present invention are directed to the provision of a pair of electrodes at a distal end of a flex circuit, such as can be used within forward facing IVL emitters as discussed above. However, it is also contemplated that flex circuits in accordance with the present invention can instead by used in other than axial firing designs, wherein the distal ends of the flex circuit conductors can comprise an electrode or may provide a bond pad or other electrical connection or connector that can be connected with any other electrode design. Such a bond pad can be used in other axially firing emitter designs, radially firing emitter designs or otherwise. As such, in the following description, the term electrode can be read as actually comprising an electrode in accordance with and IVL device or as a bond bad or other electrical connection or connector that can be connected with an electrode of an IVL device.

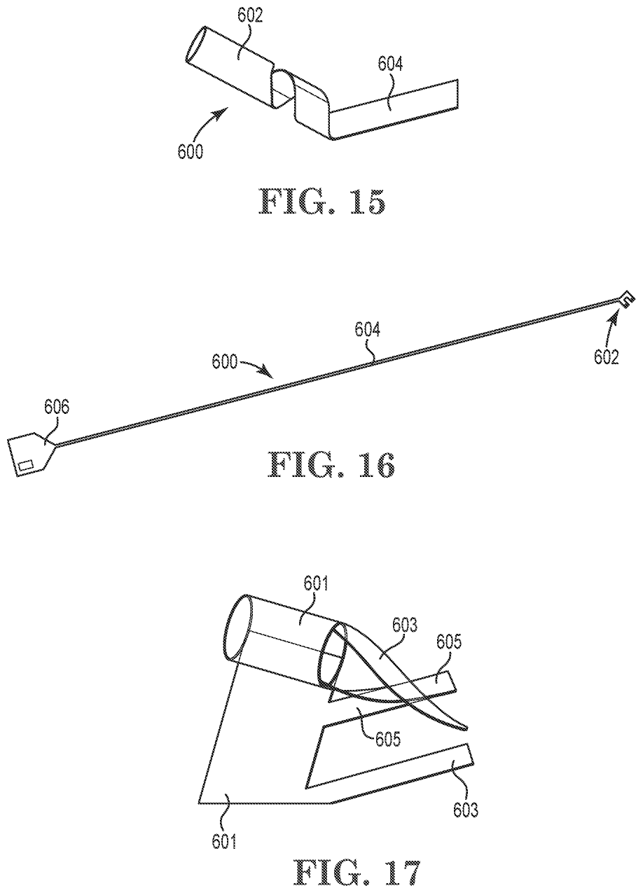

In FIGS. 15 and 16, a flex circuit 600 is illustrated, the purpose of which is to be spiral wound along the length of a catheter as par of an IVL device. A distal pad 602 is shown with an extension portion 604 extending proximally therefrom at an angle to create the spiral wind. The angle can be based on the width of the flex circuit 604 so as to wind within and along the distance of a catheter (not shown) preferably without any overlap of the windings. FIG. 15 shows an initial winding that would be located near the distal end of the flex circuit 604 and distal pad 602. The distal pad 602 and the extension portion 604 can include plural (preferably two) electrical traces and electrodes or pads, as can be manufactured by any flex circuit producing processes as discussed above or otherwise developed (discussed in greater detail below). Such electrical traces can be run to the distal end of the flex circuit to terminate at a proximal pad portion 606 for electrical connection with a high voltage pulse generator as discussed above as well. It is contemplated that a control system or module can be provided at this connection with the high voltage pulse generator as well for controlled actuation of the high voltage pulses for and IVL process.

FIG. 17 illustrates a similar concept as that of FIGS. 15 and 16 but with plural (preferably two) extension portions 603 and 605 extending at similar angles from a distal pad 601. Also illustrated are these same components in a rolled state of the distal pad 601 and the beginnings of plural windings of the extension portions 603 and 605 as they are extending within and along the length of the IVL catheter.

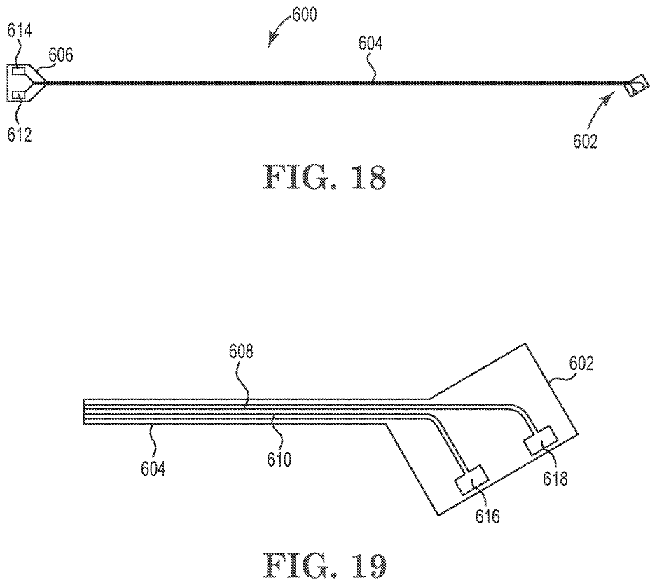

In FIGS. 18 and 19, the flex circuit 600 of FIGS. 15 and 16 is illustrated with electrical traces 608 and 610 running from proximal electrical bond pads 612 and 614 provided on the proximal pad 606 to distal electrodes or bond pads 616 and 618 provided on the distal pad 602. As such, electrical conductors are run from the proximal pad 606 to the distal pad 602, as comprising the bond pads 612 and 614, the traces 608 and 610, and the electrodes or bond pads 616 and 618, respectively. An insulator layer is also provided along with the electrical conductors, as comprising the proximal pad 606, the extension portion 604, and the distal pad 602. Preferably, the traces 608 and 610 are spaced from one another so as not to electrically interfere with one another and so as not to cause the insulation between them to breakdown during a high voltage pulse or over any amount of time of expected usage of the IVL system. Preferably, the bond pads 612 and 614 and the electrodes or bond pads 616 and 618 can be adequately spaced from one another as provided on the proximal and distal pads 606 and 602 as such pads can be larger than the width of the extension portion 604 even to accommodate larger bond pads or electrodes.

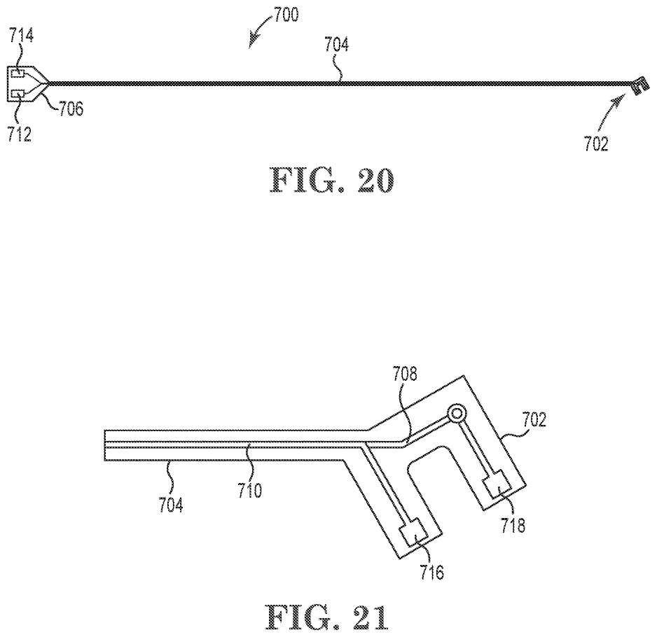

Another embodiment of a flex circuit 700 in accordance with the present invention is illustrated within FIGS. 20 and 21. Similar to the flex circuit 600, an insulating layer can comprise a proximal pad 706, and extension portion 704, and a distal pad 702. In this case, a first electrical trace 710 can be run along one side (a front side) of the insulating layer with a second electrical trace 708 run along the other side (a back side) of the insulating layer. To do this, a bond pad 712 can be provided on a first side of the proximal pad 706 that is electrically formed with the trace 710 as further electrically formed with a distal electrode or bond pad 716. An electrical trace 708 can then be run along the second side and formed or connected with a proximal bond pad 714 and a distal electrode or bond pad 718. The proximal and distal bond pads or electrodes 714 and 718 can be provided either on the second side of the insulating layer or the first side of the insulating layer. In the latter case, electrical vias can connect one or both of the proximal bond pad 714 and the distal electrode or bond pad 718 to the trace 708. In the case where the electrodes are provided at 716 and 718 for example for a forward firing arrangement, it would be preferable to have both electrodes on the same side of the distal pad 702. FIGS. 20 and 21 illustrate a top (blue) trace portion of trace 708 only partially along each proximal and distal pads 706 and 702 and as connected by electrical vias to the remainder of the trace 708 running along the second side of the insulator. The advantage of this design is better insulation between the traces 708 and 710 while allowing a narrower extension portion 704 of the flex circuit for easier winding.

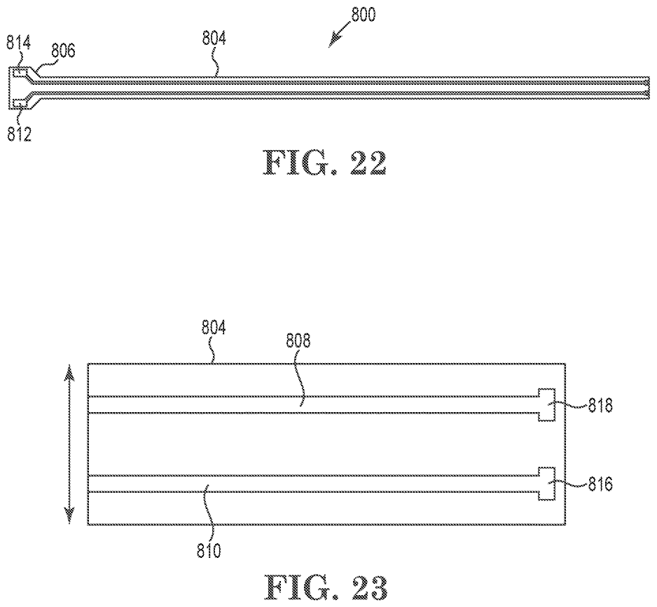

Yet another flex circuit in accordance with the present invention is illustrated in FIGS. 22 and 23. Flex circuit 800 is similar to the flex circuit 600 in FIGS. 18 and 19, but is lacking a distal pad for accommodating provision of electrodes or bond pads 816 and 818. In this embodiment, traces 808 and 810 are run side-by-side on one surface of an insulating layer along an extension portion 804 between a proximal pad 806 and a distal end. The traces 806 and 810 are spaced further from one another as they run along the extension portion 804 of the flex circuit 800 so as to provided better insulation of the traces to one another during high voltage pulsing. A greater width of the extension portion 804 provides sufficient room for both traces 808 and 810 to be spaced further apart. However, this greater width of the extension portion 804 can make it more difficult to create a spiral winding of the flex circuit 800 as positioned within and along an IVL catheter. In this case, the flex circuit 800 could simply run along the IVL catheter without winding. As illustrated in FIG. 23, electrodes can be provided at 816 and 818 that are spaced similar to the spacing of the traces 808 and 810, but need not be.

It is also understood that assemblies or sub-assemblies such as the above noted flex circuits can be made in other ways than flex circuit techniques, such as by make each of the elements separately and then assembling. Other arrangements are contemplated with two or more electrodes and any number of conductive layers or tubes. Although it is preferable that the electrode and conductive tube or layer arrangement create primarily forward or axial energy wave propagation, it is contemplated that an arrangement can create energy wave propagation that is more radial than axial, but preferably at least with an axial component.

It is also understood that the electrode and conductive tube arrangement need not be limited to a cylindrical shape. It is preferably that the electrodes are shaped to be similar to spaced portions of the conductive material as is preferably a tube or partial tube that can have a circular cross section or a portion thereof or other shapes like a square, rectangle, hexagon, etc. As above, by controlling the spacing of the electrodes to a conductive wall portion of similar shape, sparks will jump across the similar gap, and the sparks will travel along the front facing edge from side to side as the electrodes wear over time along with the conductive wall portion. Preferably, the tube or portion thereof can electrically connect the spark gaps created by the spacings in series.

1. An intravascular lithotripsy (IVL) system for use in providing an energy wave as a force to a lesion within a vasculature, the IVL system comprising,
a catheter that extends from a proximal end to a distal end with an electrode and conductive tube arrangement at a distal end of the catheter, wherein the electrode is provided within the conductive tubing and spaced from the conductive tubing by an insulating layer, the electrode having a forward-facing electrode distal end, the conductive tubing also including a forward-facing conductive tubing distal end that is spaced radially relative to the distal end of the electrode to create at least one spark gap between the forward facing electrode distal end.

2. The IVL system of claim 1, wherein plural electrodes are provided within the conductive tubing, each spaced from the conductive tubing by the insulating layer and with the electrodes being electrically isolated from one another so as to create plural gaps with the conductive tubing that can be energized in series to create plural sparks.

3. The IVL system of claim 2, wherein the electrodes are arc segments of conductive material that as spaced similarly from the conductive tubing are coaxial with the conductive tubing, and the distal ends of the electrodes and the distal end of the conductive tubing extend to a similar axial length so as to terminated adjacent to one another.

4. The IVL system of claim 4, wherein the insulating layer similarly extends to terminate adjacent to the distal ends of the electrodes and the conductive tubing, and a guide wire insulating layer is provided inside of the electrodes to define a guidewire lumen.

5. The IVL system of claim 1, wherein the distal end of the conductive tubing extends axially more distally than the distal end of the electrode so that the entire distal end of the electrode is proximally positioned within the conductive tubing and spaced from the distal end of the conductive tubing.

6. The IVL system of claim 5, wherein the insulating layer extends to terminate adjacent to the distal end of the electrode so that a spark can be generated between the distal end of the electrode and an inside side wall of the conductive tubing.

7. The IVL system of claim 1, wherein the electrode and insulating layer comprise a flex circuit that can be rolled into a cylinder and inserted within the conductive tubing.

8. The IVL system of claim 7, wherein the conductive tubing is also created as a part of the flex circuit.

9. A method of using an intravascular lithotripsy (IVL) system against a lesion within a vasculature comprising:
inserting an IVL catheter having a balloon, a conductive tubing, and at least one electrode within the vasculature of a patient to the point of a lesion within the vasculature being located beyond the distal end of the balloon, wherein the electrode has a distal end and the conductive tube also has a distal end so that a spark gap is created between the distal ends of the electrode and the conductive tubing;
electrically connecting the conductive tubing and the electrode with a high voltage pulse generator;
delivering a fluid that is at least partially conductive to the balloon; and
generating a high voltage pulse at the high voltage pulse generator and thereby creating a spark at the spark gap with a cavitation bubble such that forward-facing energy waves are propagated within the fluid of the balloon and from the balloon to the lesion.

10. The IVL system of claim 9, wherein plural electrodes are provided within the conductive tubing, each spaced from the conductive tubing by the insulating layer and with the electrodes being electrically isolated from one another so as to create plural spark gaps with the conductive tubing, the method further comprising energizing the electrodes in series to create plural sparks at the plural spark gaps.

11. The IVL system of claim 10, wherein the distal end of the electrodes and the distal end of the conductive tubing extend axially a similar distance and terminate adjacent to one another with the insulating layer between them so that the spark can be generated from the distal end of the electrodes and the distal end of the conductive tubing.

12. The IVL system of claim 9, wherein the distal end of the conductive tubing extends axially more distally than the distal end of the electrode so that the entire distal end of the electrode is proximally positioned within the conductive tubing and spaced from the distal end of the conductive tubing, the method further comprising generating a spark between a distal end of the electrode and an inside side wall of the conductive tubing and thus generating a cavitation bubble at least partially within the distal end of the conductive tubing so that energy waves can be directed from an open distal end of the conductive tubing in a desired forward direction.

13. The IVL system of claim 12, wherein the distal end of the conductive tubing extends sufficiently axially beyond the distal end of the electrode so that the entire cavitation bubble is formed within the distal end of the conductive tubing.

14. An intravascular lithotripsy (IVL) system for use in providing an energy wave as a force to a lesion with a vasculature, the IVL system comprising,
a catheter that extends from a proximal end to a distal end with an electrode and conductive tube arrangement at a distal end of the catheter with plural electrodes provided adjacent to the distal end of the catheter, also including a flex circuit extending from the proximal end of the catheter to the electrodes for electrically connecting the electrodes to a high voltage pulse generator at the proximal end of the flex circuit, wherein the flex circuit is spirally wound within and along at least a portion of the catheter.

15. The IVL system of claim 14, wherein the electrodes are formed on a flex pad portion of the flex circuit at the distal end thereof, and an extension portion of the flex circuit extends from the flex pad proximally within the catheter, the extension portion of the flex circuit also having plural conductive traces running along the extension portion.

16. The IVL system of claim 15, wherein the extension portion extends from the flex pad at an angle so that the traces are arrange at that angle relative to the positioning of the electrodes to facilitate spirally winding of the extension portion within and along the catheter.

17. The IVL system of claim 15, wherein plural traces are provided along one side surface of the extension portion of the flex circuit.

18. The IVL system of claim 15, wherein at least one trace is provided to run along one side surface of the extension portion of the flex circuit and at least one other trace is run along a second side surface of the extension portion of the flex circuit.
